SEQUENCE LISTING (31 sequences)

SEQ ID NO: 1 - protein, 20 aa, Artificial Sequence
  Synthetic Peptide
  misc_feature (7)..(7): Xaa can be any naturally occurring amino acid or non-genetically encoded amin acid
  misc_feature (9)..(12): Xaa can be any naturally occurring amino acid or non-genetically encoded amin acid
  misc_feature (18)..(18): Xaa can be any naturally occurring amino acid or non-genetically encoded amin acid
  misc_feature (18)..(18): Xaa can be any naturally occurring amino acid or non-genetically encoded amino acid
LRRFSTXPXXXXNINNVXNF

SEQ ID NO: 2 - protein, 20 aa, Artificial Sequence
  Synthetic Peptide
LRRFSTAPFAFIDINDVINF

SEQ ID NO: 3 - protein, 20 aa, Artificial Sequence
  SYNTHETIC PEPTIDE
LRRFSTAPFAFININNVINF

SEQ ID NO: 4 - protein, 20 aa, Artificial Sequence
  SYNTHETIC PEPTIDE
LRRFSTAPFAFIDINDVINW

SEQ ID NO: 5 - protein, 9 aa, Artificial Sequence
  SYNTHETIC PEPTIDE
FTNINNVTN

SEQ ID NO: 6 - protein, 9 aa, Artificial Sequence
  SYNTHETIC PEPTIDE
FTDINDVTN

SEQ ID NO: 7 - protein, 20 aa, Artificial Sequence
  SYNTHETIC PEPTIDE
  Non-genetically encoded amin acid (12)..(12): Abu (2-Aminobutyric acid)
  Non-genetically encoded amin acid (18)..(18): Abu (2-Aminobutyric acid)
LRRFSTMPFMFXNINNVXNF

SEQ ID NO: 8 - protein, 20 aa, Artificial Sequence
  SYNTHETIC PEPTIDE
  Non-Genetically encoded amin acid (12)..(12): Abu (2-Aminobutyric acid)
  Non-Genetically encoded amin acid (18)..(18): Abu (2-Aminobutyric acid)
LRRFSTMPAMFXNINNVXNF

SEQ ID NO: 9 - protein, 20 aa, Artificial Sequence
  SYNTHETIC PEPTIDE
  Non-Genetically encoded amin acid (12)..(12): Abu (2-Aminobutyric acid)
  Non-Genetically encoded amin acid (18)..(18): Abu (2-Aminobutyric acid)
LRRFSTMPFAFXNINNVXNF

SEQ ID NO: 10 - protein, 20 aa, Artificial Sequence
  SYNTHETIC PEPTIDE
  Non-Genetically encoded amin acid (12)..(12): Abu (2-Aminobutyric acid)
  Non-Genetically encoded amin acid (18)..(18): Abu (2-Aminobutyric acid)
LRRFSTMPFMAXNINNVXNF

SEQ ID NO: 11 - protein, 20 aa, Artificial Sequence
  SYNTHETIC PEPTIDE
  Non-Genetically encoded amin acid (10)..(10): Nle (norleucine)
  Non-Genetically encoded amin acid (12)..(12): Abu (2-Aminobutyric acid)
  Non-Genetically encoded amin acid (18)..(18): Abu (2-Aminobutyric acid)
LRRFSTMPFXFXNINNVXNF

SEQ ID NO: 12 - protein, 20 aa, Artificial Sequence
  SYNTHETIC PEPTIDE
  Non-Genetically encoded amin acid (11)..(11): 4-ClPhe (4-chlorophenylalanine)
  Non-Genetically encoded amin acid (12)..(12): Abu (2-Aminobutyric acid)
  Non-Genetically encoded amin acid (18)..(18): Abu (2-Aminobutyric acid)
LRRFSTMPFMXXNINNVXNF

SEQ ID NO: 13 - protein, 20 aa, Artificial Sequence
  SYNTHETIC PEPTIDE
LRRFSTMPFMFSNINNVSNF

SEQ ID NO: 14 - protein, 20 aa, Artificial Sequence
  SYNTHETIC PEPTIDE
LRRFSTMPFMFANINNVANF

SEQ ID NO: 15 - protein, 20 aa, Artificial Sequence
  SYNTHETIC PEPTIDE
LRRFSTMPFMFININNVINF

SEQ ID NO: 16 - protein, 20 aa, Artificial Sequence
  SYNTHETIC PEPTIDE
LRRFSTMPFMFTNINNVTNF

SEQ ID NO: 17 - protein, 20 aa, Artificial Sequence
  SYNTHETIC PEPTIDE
  Non-Genetically encoded amin acid (12)..(12): AllylGly (Allylglycine)
  Non-Genetically encoded amin acid (18)..(18): AllylGly (Allylglycine)
LRRFSTMPFMFXNINNVXNF

SEQ ID NO: 18 - protein, 20 aa, Artificial Sequence
  SYNTHETIC PEPTIDE
LRRFSTMPFMFVNINNVVNF

SEQ ID NO: 19 - protein, 20 aa, Artificial Sequence
  SYNTHETIC PEPTIDE
  Non-Genetically encoded amin acid (10)..(10): d Alanine
LRRFSTMPFXFININNVINF

SEQ ID NO: 20 - protein, 20 aa, Artificial Sequence
  SYNTHETIC PEPTIDE
LRRFSTMPFAFININNVINF

SEQ ID NO: 21 - protein, 20 aa, Artificial Sequence
  SYNTHETIC PEPTIDE
LRRFSTAPFAFININNVINF

SEQ ID NO: 22 - protein, 20 aa, Artificial Sequence
  SYNTHETIC PEPTIDE
  Non-Genetically encoded amin acid (10)..(10): d Alanine
LRRFSTAPFXFIDINDVINF

SEQ ID NO: 23 - protein, 9 aa, Artificial Sequence
  SYNTHETIC PEPTIDE
  Non_Genetically encoded amin acid (2)..(2): Abu (2-Aminobutyric acid)
  Non_Genetically encoded amin acid (8)..(8): Abu (2-Aminobutyric acid)
FXNINNVXN

SEQ ID NO: 24 - protein, 9 aa, Artificial Sequence
  SYNTHETIC PEPTIDE
FTNINNVTN

SEQ ID NO: 25 - protein, 10 aa, Artificial Sequence
  SYNTHETIC PEPTIDE
FININNVINF

SEQ ID NO: 26 - protein, 10 aa, Artificial Sequence
  SYNTHETIC PEPTIDE
FSNINNVSNF

SEQ ID NO: 27 - protein, 10 aa, Artificial Sequence
  SYNTHETIC PEPTIDE
FANINNVANF

SEQ ID NO: 28 - protein, 10 aa, Artificial Sequence
  SYNTHETIC PEPTIDE
  Non-Genetically encoded amin acid (2)..(2): AllylGly (Allylglycine)
  Non-Genetically encoded amin acid (8)..(8): AllylGly (Allylglycine)
FXNINNVXNF

SEQ ID NO: 29 - protein, 10 aa, Artificial Sequence
  SYNTHETIC PEPTIDE
FVNINNVVNF

SEQ ID NO: 30 - protein, 10 aa, Artificial Sequence
  SYNTHETIC PEPTIDE
  Non-Genetically encoded amin acid (2)..(2): Abu (2-Aminobutyric acid)
  Non-Genetically encoded amin acid (8)..(8): Abu (2-Aminobutyric acid)
AXNINNVXNF

SEQ ID NO: 31 - protein, 10 aa, Artificial Sequence
  SYNTHETIC PEPTIDE
  Non-Genetically encoded amin acid (1)..(1): 4-ClPhe (4-chlorophenylalanine)
  Non-Genetically encoded amin acid (2)..(2): Abu (2-Aminobutyric acid )
  Non-Genetically encoded amin acid (8)..(8): Abu (2-Aminobutyric acid )
XXNINNVXNF

(19) United States
(12) Patent Application Publication
(10) Pub. No.: US 2018/0339024 A1
(43) Pub. Date: Nov. 29, 2018

(54) PEPTIDES WITH ANTI-ANGIOGENIC, ANTI-LYMPHANGIOGENIC, AND ANTI-EDEMIC PROPERTIES AND NANOPARTICLE FORMULATIONS

(71) Applicant: Asclepix Therapeutics, LLC, Baltimore, MD (US)

(72) Inventors: Eric M. BRESSLER, Baltimore, MD (US); Jordan J. GREEN, Baltimore, MD (US); Niranjan B. PANDEY, Baltimore, MD (US); Aleksander S. POPEL, Baltimore, MD (US); Ron B. SHMUELI, Sr., Baltimore, MD (US)

(21) Appl. No.: 15/776,971

(22) PCT Filed: Nov. 18, 2016
(86) PCT No.: PCT/US16/62816

§ 371 Date: May 17, 2018

Related U.S. Application Data
(60) Provisional application No. 62/257,569, filed on Nov. 19, 2015.

(51) Int. Cl. A61K 38/39; A61K 47/69; A61K 9/50; A61K 9/00; G01N 33/68; A61K 45/06; A61P 27/02; A61P 35/00

(52) U.S. Cl. CPC A61K 38/39; A61K 47/6937; A61K 9/50; A61K 9/0048; G01N 2333/70546; A61K 45/06; A61P 27/02; A61P 35/00; G01N 2333/78; G01N 33/6887

(57) ABSTRACT

The present invention in various aspects and embodiments involves pharmaceutical compositions of peptides derived from the α5 fibril of type IV collagen, and uses thereof for medical treatment. The peptides target α5β1 and αVβ3 integrins, and inhibit signaling through multiple receptors, and find use for inhibiting vascular permeability, angiogenesis, lymphangiogenesis.

### PRIORITY

This application claims the benefit of, and priority to, U.S. Provisional Application No. 62/257,569, filed Nov. 19, 2015, which is hereby incorporated by reference in its entirety.

### BACKGROUND

Peptides derived from collagen type IV have been described for their potential to inhibit angiogenesis and lymphangiogenesis. Peptide motifs derived from the non-collagenous domain of the α5 fibril of type IV collagen are described in U.S. Pat. No. 9,056,923, which is hereby incorporated by reference in its entirety. For example, a peptide comprising the motif NINNV is described as inhibiting proliferation, migration, and tubule formation of human umbilical vein endothelial cells (HUVEC). Rosca et al., Structure-activity relationship study of collagen derived anti-angiogenic biomimetic peptides, Chem. Biol. Drug Des. 80(1):27-37 (2012).

A better understanding of the biological targets, biological activities, and pharmaceutical properties of these peptides are needed to support and/or direct pharmaceutical uses and product development.

### BRIEF DESCRIPTION

The present invention in various aspects and embodiments involves pharmaceutical compositions of peptides derived from the α5 fibril of type IV collagen, and uses thereof for medical treatment. Exemplary peptides comprise the amino acid sequence LRRFSTAPFAFIDINDVINF (SEQ ID NO:2), LRRFSTAPFAFININNVINF (SEQ ID NO:3), LRRFSTAPFAFIDINDVINW (SEQ ID NO:4), FTNINNVTN (SEQ ID NO:5), or FTDINDVTN (SEQ ID NO:6), or an amino acid sequence that is a derivative of any of the foregoing, including various derivatives described herein. The peptides target α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and platelet-derived growth factor receptor (PDGFR).

In some aspects, the invention provides a method for treating or preventing microvascular leakage or permeability, comprising administering an effective amount of the peptide agent to a patient in need of treatment. Conditions where microvascular leakage is involved in or can exacerbate a pathology include influenza (flu), Alzheimer's disease, neuropathologies (e.g., multiple sclerosis), hemorrhagic fever, cerebral malaria, macular degeneration, macular edema (e.g., diabetic macular edema), retinal vein occlusion (RVO), diabetic retinopathy, wet AMD, acute respiratory distress syndrome, pulmonary edema, asthma, COPD, Respiratory Syncytial Virus, SARS, pneumonia, microvascular leakage and vascular permeability associated with tissue or organ transplantation, among others.

In other aspects, the invention provides a method for treating cancer, and in particular for improving checkpoint inhibitor therapy. Specifically, the method in these embodiments improves immune checkpoint inhibitor therapy by inhibiting angiogenesis and allowing dendritic cell maturation and more robust lymphocyte endothelial trafficking. The method comprises administering an effective amount of the peptide to a cancer patient undergoing therapy with an immune checkpoint inhibitor. The peptide or pharmaceutical composition may be administered with (or during) immune checkpoint inhibitor therapy. Alternatively, the patient may be treated for one to four weeks with the peptide, followed by immune checkpoint inhibitor therapy.

The peptide may be formulated for systemic delivery or local delivery, and in some embodiments, the peptide is formulated with a polymeric nanoparticle or microparticle carrier. In some embodiments, the invention provides a nanoparticle comprising PLGA-PEG copolymers and a conjugated peptide targeting integrins, such as the peptide of any one of SEQ ID NO: 1 to 6, or derivatives and/or combinations thereof. In some embodiments, the nanoparticle is synthesized from poly(lactic-co-glycolic acid) polyethylene glycol (PLGA-PEG) block copolymers of tunable size which are covalently linked to the peptide, or other binding agent as described above. A mix of conjugated and unconjugated polymers in various ratios can create nanoparticles with the desired density of targeting agent on the surface. The particles may be designed to provide desired pharmacodynamic advantages, including circulating properties, biodistribution, and degradation kinetics. Such parameters include size, shape, surface charge, polymer composition, ligand conjugation chemistry, peptide conjugation density, among others.

In some embodiments, the nanoparticle further comprises an encapsulated active agent, which may be an active agent disclosed herein for treatment of conditions characterized by microvascular leakage, including flu, Alzheimer's Disease, hemorrhagic fever, cerebral malaria, cancer, macular degeneration or macular edema, organ or tissue transplantation, and others described herein. In some embodiments, the encapsulated agent is a peptide described herein. While the nanoparticle is substantially spherical in some embodiments, the nanoparticle may optionally be non-spherical to affect its interactions with cells, and particularly with cells of the immune system to avoid clearance.

In some embodiments, the particle is a microparticle that encapsulates a drug cargo (such as a peptide described herein, and/or other agent). The particle may or may not contain peptide conjugated to the surface. In these embodiments, the particle can provide a long acting drug depot, to provide a sustained release of peptide.

Other aspects and embodiments of the invention will be apparent from the following detailed description.

### BRIEF DESCRIPTION OF THE DRAWINGS

FIG. 1 shows that P07 inhibits VEGF, HGF, and IGF signaling in microvascular endothelial cells.

FIG. 2 shows that P07 inhibits retinal detachment caused by excessive vascular leakage in a mouse model.

FIG. 3 shows that P07 inhibits vascular leakage in a VEGF induced leakage model in the rabbit eye. Leakage is compared to untreated, which is set at 1.0.

FIG. 4 shows that P07 inhibits the growth of orthotopic triple negative breast cancer (TBNC) xenografts in a dose dependent manner.

FIG. 5 shows that P07 inhibits neovascularization in orthotopic TNBC xenografts.

FIG. 6 shows the HPLC results for P08 conjugation, demonstrating that P08 was conjugated efficiently to PLGA-PEG-NHS copolymers and quantified via reverse phase HPLC.

FIG. 7 shows that N07 exhibits a Z-average diameter of approximately 70-80 nm. Slight size increase is seen in samples with conjugated P07 on the nanoparticle.

FIG. 8 shows N07 exhibits a negative zeta potential in deionized water, which is very slightly tunable around −25 mV using different end groups on PEG. Neutral is methoxy-terminated PEG. Negative is carboxy-terminated PEG.

FIG. 9 shows the binding of particles to integrin αvβ3 and α5β1.

FIG. 10 shows the binding of particles to integrin αvβ3 with competition from various peptides.

FIG. 11 shows the binding of N07 particles to MDA-MB-231 and MEC cells.

FIG. 12 shows adhesion inhibition assay results measuring the adhesion of cells to plates pre-treated with N07. % refers to amount of PLGA-PEG molecules that have conjugated P07. “+” or “−” refers to the presence or absence of encapsulated P07.

FIG. 13 shows the inhibitory effects of N07 on proliferation of MEC cells. % refers to amount of PLGA-PEG molecules that have conjugated P07. “+” or “−” refers to the presence or absence of encapsulated P07.

FIG. 14 shows adhesion assay results measuring the adhesion of cells to plates pre-treated with P07.

FIG. 15 shows microparticles (MPs) made with 85/15 PLGA and P07, also called M07, using double emulsion technique. Lyophilized samples were imaged with SEM. (A) 0% loading; (B) 0.6% final peptide loading by weight; (C) 1% final loading. Scale shown is 10 μM.

FIG. 16 shows M07, stretched 2.25×. Lyophilized samples were imaged with SEM. (A) blank MPs; (B) M07; (C) blank MPs, zoomed in; (D) M07, zoomed in.

FIG. 17 shows TEM images of peptide loaded NPs nonstretched (left) or 2× stretched (right).

FIG. 18 shows Gel quantification of P07 loading in stretched MPs. Quantified using silver staining and peptide standard. Final P07 w/w ratio ˜1%, which is comparable to the pre-stretching P07 w/w ratio of −1%.

FIG. 19 shows release of P07 from 65/35 PLGA microparticles loaded with P07. Error bars represent standard deviation.

### DETAILED DESCRIPTION

The present invention in various aspects and embodiments involves pharmaceutical compositions of peptides derived from the α5 fibril of type IV collagen, and uses thereof for medical treatment. Exemplary peptides comprise the amino acid sequence LRRFSTAPFAFIDINDVINF (SEQ ID NO:2), LRRFSTAPFAFININNVINF (SEQ ID NO:3), LRRFSTAPFAFIDINDVINW (SEQ ID NO:4), FTNINNVTN (SEQ ID NO:5), or FTDINDVTN (SEQ ID NO:6), or an amino acid sequence that is a derivative of any of the foregoing. Various derivatives are disclosed herein. The peptides target α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and platelet-derived growth factor receptor (PDGFR).

Peptides targeting integrins include those described in U.S. Pat. No. 9,056,923, which is hereby incorporated by reference in its entirety. For example, peptides in accordance with the following disclosure include peptides comprising the amino acid sequence LRRFSTXPXXXXNINNVXNF (SEQ ID NO:1), where X is a standard amino acid or non-genetically encoded amino acid. In some embodiments, X at position 7 is M, A, or G; X at position 9 is F, A, Y, or G; X at position 10 is M, A, G, dA, or Nle; X at position 11 is F, A, Y, G, or 4-ClPhe; X at position 12 and position 18 are independently selected from Abu, G, S, A, V, T, I, L or Allyl-Gly. In various embodiments, the peptide contains about 30 amino acids or less, or about 25 amino acids of less, or about 24 amino acids, or about 23 amino acids, or about 22 amino acids, or about 21 amino acids, or about 20 amino acids. In still other embodiments, from one to ten amino acids, such as one, two or three amino acids of SEQ ID NO:1 are deleted. For example, amino acids from N-terminus are deleted in some embodiments.

In some embodiments, the peptide comprises the amino acid sequence LRRFSTAPFAFIDINDVINF (SEQ ID NO:2), or LRRFSTAPFAFININNVINF (SEQ ID NO:3), or LRRFSTAPFAFIDINDVINW (SEQ ID NO:4), or FTNINNVTN (SEQ ID NO:5), or FTDINDVTN (SEQ ID NO:6), or an amino acid sequence that is a derivative of any of the foregoing. The peptide of SEQ ID NO:2 is also referred to herein as P07. The peptide of SEQ ID NO:3 is also referred to herein as P06. The peptide of SEQ ID NO:4 is also referred to herein as P08. The peptide of SEQ ID NO:5 is also referred to herein as P05. The peptide of SEQ ID NO:6 is also referred to herein as P09. The peptide of SEQ ID NO:2 (in comparison to SEQ ID NO:1) has an Aspartic Acid at positions 13 and 16, which improves the physical properties of the peptide without negatively impacting the biological activities. Derivatives of the peptides of SEQ ID NOS:2 to 4 include peptides having from 1 to 5 amino acid substitutions, insertions, or deletions (e.g., 1, 2, 3, 4, or 5 amino acid substitutions, insertions, or deletions collectively) with respect to SEQ ID NO:2, 3, or 4. In some embodiments, the Asp at positions 13 and 16 of SEQ ID NO: 2 is maintained. In some embodiments, the sequence DINDV or NINNV is maintained in the derivative. Amino acid substitutions can optionally be at positions occupied by an X at the corresponding position of SEQ ID NO:1. The peptide generally has at least 8 amino acids. Derivatives of peptides of SEQ ID NO: 5 or 6 include peptides comprising a sequence having 1, 2, or 3 amino acid substitutions with respect to the sequence of SEQ ID NO: 5 or 6. In some embodiments, amino acid substitutions are independently selected from conservative or non-conservative substitutions. In these or other embodiments, the peptide includes from 1 to 10 amino acids added to one or both termini (collectively). The N- and/or C-termini may optionally be occupied by another chemical group (other than amine or carboxy, e.g., amide or thiol), and which can be useful for conjugation of other moieties, including PEG or PLGA-PEG co-polymers, as described in further detail herein. Peptides may be provided in the form of a pharmaceutically acceptable salt in some embodiments, or complexed with other components or encapsulated in particles for targeted or sustained delivery to particular tissues.

Conservative substitutions may be made, for instance, on the basis of similarity in polarity, charge, size, solubility, hydrophobicity, hydrophilicity, and/or the amphipathic nature of the amino acid residues involved. The 20 genetically encoded amino acids can be grouped into the following six standard amino acid groups:

(1) hydrophobic: Met, Ala, Val, Leu, Ile;

(2) neutral hydrophilic: Cys, Ser, Thr; Asn, Gln;

(3) acidic: Asp, Glu;

(4) basic: His, Lys, Arg;

(5) residues that influence chain orientation: Gly, Pro; and

(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

In various embodiments, the peptide agent is a peptide of from about 8 to about 30 amino acids, or from about 10 to about 20 amino acids, and has at least 4, at least 5, or at least 6 contiguous amino acids of SEQ ID NO:2, 3, 4, 5, or 6. In some embodiments, the peptide contains at least one, at least two, or at least three D-amino acids. In some embodiments, the peptide contains from one to about five (e.g., 1, 2, or 3) non-genetically encoded amino acids, which are optionally selected from 2-Aminobutyric acid (Abu), norleucine (Nle), 4-chlorophenylalanine (4-ClPhe), and Allylglycine (AllylGly).

Exemplary peptide agents, which may be derivatives of the peptides of SEQ ID NOS: 2 to 6 in accordance with the disclosure, include:

(SEQ ID NO: 7)
LRRFSTMPFMF(Abu)NINNV(Abu)NF,
(SEQ ID NO: 8)
LRRFSTMPAMF(Abu)NINNV(Abu)NF,
(SEQ ID NO: 9)
LRRFSTMPFAF(Abu)NINNV(Abu)NF,
(SEQ ID NO: 10)
LRRFSTMPFMA(Abu)NINNV(Abu)NF,
(SEQ ID NO: 11)
LRRFSTMPF(Nle)F(Abu)NINNV(Abu)NF,
(SEQ ID NO: 12)
LRRFSTMPFM(4-ClPhe)(Abu)NINNV(Abu)NF,
(SEQ ID NO: 13)
LRRFSTMPFMFSNINNVSNF,
(SEQ ID NO: 14)
LRRFSTMPFMFANINNVANF,
(SEQ ID NO: 15)
LRRFSTMPFMFININNVINF,
(SEQ ID NO: 16)
LRRFSTMPFMFTNINNVTNF,
(SEQ ID NO: 17)
LRRFSTMPFMF(AllyGly)NINNV(AllyGly)NF,
(SEQ ID NO: 18)
LRRFSTMPFMFVNINNVVNF,
(SEQ ID NO: 19)
LRRFSTMPFdAFININNVINF,
(SEQ ID NO: 20)
LRRFSTMPFAFININNVINF,
(SEQ ID NO: 21)
LRRFSTAPFAFININNVINF,
(SEQ ID NO: 22)
LRRFSTAPFdAFIDINDVINF,
(SEQ ID NO: 23)
F(Abu)NINNV(Abu)N,
(SEQ ID NO: 24)
FTNINNVTN,
(SEQ ID NO: 25)
FININNVINF,
(SEQ ID NO: 26)
FSNINNVSNF,
(SEQ ID NO: 27)
FANINNVANF,
(SEQ ID NO: 28)
F(AllyGly)NINNV(AllyGly)NF,
(SEQ ID NO: 29)
FVNINNVVNF,
(SEQ ID NO: 30)
A(Abu)NINNV(Abu)NF,
or
(SEQ ID NO: 31)
(4-ClPhe)(Abu)NINNV(Abu)NF.

In various aspects and embodiments described herein, the peptide may be delivered in the form of nanoparticle and microparticle formulations, either conjugated to the surface or encapsulated. Exemplary particle formulations based on PLGA-PEG polymers are described in detail herein.

The peptide agents can be chemically synthesized and purified using well-known techniques, such as solid-phase synthesis. See U.S. Pat. No. 9,051,349, which is hereby incorporated by reference in its entirety.

The peptides described herein target α5β1 and αVβ3 integrins, and inhibit signaling through multiple receptors, including vascular endothelial growth factor receptor (VEGFR), hepatocyte growth factor receptor (HGFR), insulin-like growth factor receptor (IGFR), and platelet derived growth factor receptor (PDGFR). Integrins are transmembrane receptors that are the bridges for cell-cell and cell-extracellular matrix (ECM) interactions. Signal transduction from integrins effects the chemical composition and mechanical status of the ECM, which controls numerous biological responses such as regulation of the cell cycle, cell shape, and/or motility; or new receptors being added to the cell membrane. This allows rapid and flexible responses to events at the cell surface. There are several types of integrins, and a cell may have several types on its surface. Integrins work alongside other receptors such as cadherins, the immunoglobulin superfamily cell adhesion molecules, selectins and syndecans to mediate cell-cell and cell-matrix interaction. Ligands for integrins include fibronectin, vitronectin, collagen, and laminin.

In some aspects, the invention provides a method for treating or preventing microvascular leakage or permeability, comprising administering an effective amount of the peptide having the amino acid sequence of any one of SEQ ID NO:1 to 6, or a derivative and/or combination thereof as described, to a patient in need of treatment. Vascular permeability, often in the form of capillary permeability or microvascular permeability, characterizes the capacity of a blood vessel wall to allow for the flow of small molecules (ions, water, nutrients) or even whole cells in and out of the vessel. Blood vessel walls are lined by a single layer of endothelial cells. The gaps between endothelial cells, known as tight junctions, are strictly regulated depending on the type and physiological state of the tissue. Increases in vascular permeability can result in edema, a condition characterized by an excess of fluid collecting in the cavities or tissues of the body.

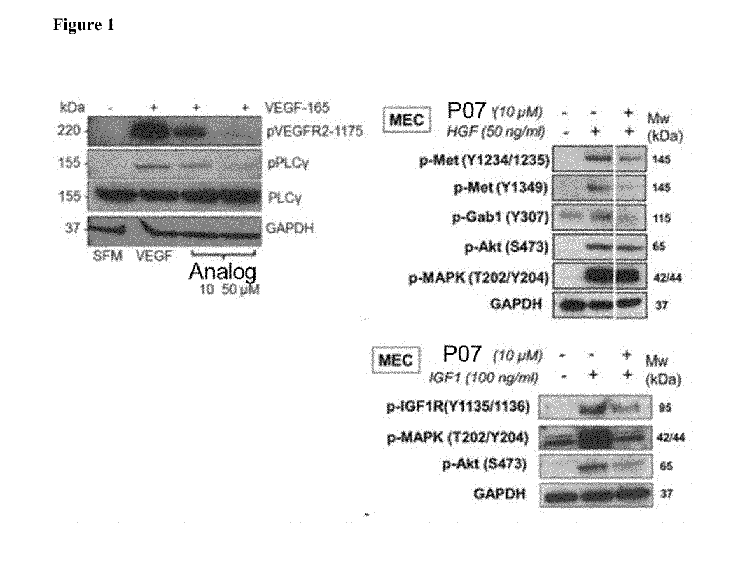

The microvascular endothelium responds to inflammatory and other stimulus, which can play a pivotal role in the pathology of many medical conditions. The potential mediators of vascular permeability, including soluble factors and cellular receptors, and their potential roles and interactions are complex, and can depend on the tissue and particular pathology. For example, microvascular leak may play a role in the pathology of influenza (flu), Alzheimer's disease, hemorrhagic fever, cerebral malaria, macular degeneration, macular edema, Retinal Vein Occlusion, diabetic retinopathy, acute respiratory distress syndrome, pulmonary edema, asthma, COPD, Respiratory Syncytial Virus, SARS, pneumonia, or vascular permeability associated with organ or tissue transplantation or cancer, among others. The peptide described herein can help treat these conditions by inhibiting signaling through multiple receptors involved in microvascular permeability, including vascular endothelial growth factor receptor (VEGFR), hepatocyte growth factor receptor (HGFR), insulin-like growth factor receptor (IGFR), and platelet derived growth factor receptor (PDGFR). See FIG. 1.

In some embodiments, the peptide or composition described herein is administered locally to the lungs, skin, or eyes, to prevent or reduce microvascular leakage or permeability.

In some embodiments, the patient has or is at risk of flu. Influenza (“the flu”) is an infectious disease caused by the influenza virus. Symptoms include a high fever, runny nose, sore throat, muscle pains, headache, coughing, and fatigue. These symptoms typically begin two days after exposure to the virus. The infection may be confirmed by testing the throat, sputum, or nose for the presence of the virus. Yearly vaccinations against influenza are recommended by the World Health Organization for those at high risk, and the vaccine is typically effective against three or four types of influenza. Antiviral drugs, such as the neuraminidase inhibitors (e.g., oseltamivir, among others) have been used to treat influenza, and while they have shown modest benefits, they must be used early in the infection (e.g., soon after symptoms appear) to provide benefit. Approximately 33% of people with influenza are asymptomatic. Symptoms of influenza can start quite suddenly around one to two days after infection. Usually the first symptoms are chills or a chilly sensation, but fever is also common early in the infection. Anti-viral treatments, although sometimes providing modest benefits, run the risk of viral resistance, which would be particularly problematic in a potent pandemic strain.

An attractive alternative to treating the virus is to treat the host response, which is much less likely to result in resistance to the drug, and may provide a greater window of efficacy in allowing treatment of more advanced stages of the illness. One of the major responses by the host is an inflammatory response that causes pulmonary microvascular leak and lung injury sometimes leading to respiratory failure. Anti-edemic agents that inhibit microvascular leak could ameliorate the symptoms of the flu.

In some embodiments, the peptide or pharmaceutical composition comprising the same is first administered before the appearance of flu symptoms. For example, the patient may be diagnosed as having flu using a laboratory test that detects the presence of the virus in patient samples, or the patient is at risk of flu after being exposed to the virus. Exposure can be determined by close contact with infected and/or symptomatic individuals.

In other embodiments, the peptide or pharmaceutical composition is first administered after first flu symptoms appear. In some embodiments, the peptide or pharmaceutical composition is administered within 1 to 4 days (such as 1 or 2 days) after the appearance of first flu symptoms.

In accordance with this aspect of the invention, the peptide reduces edema in the lung associated with influenza virus, thereby ameliorating the symptoms and/or severity of the condition. In some embodiments, the overall length of the illness can be reduced by one, two, three, four, or more days, and/or the severity and discomfort can be substantially reduced.

For treatment of a patient having or at risk of flu, the peptide or pharmaceutical composition described herein can be administered from about 1 to about 5 times daily, such as from about 1 to about 3 times daily. In some embodiments, the peptide or pharmaceutical composition is administered locally to the lungs, for example, by powder or solution aerosol, or in other embodiments is administered systemically.

In some embodiments, the peptide is administered with one or more anti-viral agents that are active against influenza, or alternatively is administered with one or more anti-inflammatory agents, either as a separate drug formulations or as a co-formulated product. Exemplary anti-viral agents include Tamiflu (oseltamivir phosphate), Relenza (zanamivir), Rapivab (peramivir), amantadine, and rimantadine. Anti-inflammatory agents include NSAIDs such as aspirin, ibuprofren, acetaminophen, and naproxen.

In other embodiments, the peptide or pharmaceutical composition is administered for the treatment of, or to slow the progression of, Alzheimer's disease. The blood-brain barrier (BBB) limits entry of blood-derived products, pathogens, and cells into the brain that is essential for normal neuronal functioning and information processing. Post-mortem tissue analysis indicates BBB damage in Alzheimer's disease. The timing of BBB breakdown remains, however, elusive. Advanced dynamic contrast-enhanced MRI with high spatial and temporal resolutions to quantify regional BBB permeability in the living human brain have shown an age-dependent BBB breakdown in the hippocampus, a region critical for learning and memory that is affected early in AD. These data suggest that BBB breakdown is an early event in the aging human brain that begins in the hippocampus and may contribute to cognitive impairment. Thus, an agent that inhibits blood-brain damage and the resulting increased permeability could slow down the progress of Alzheimer's disease. Administration of the peptide or compositions described herein in some embodiments, maintain the integrity of the blood-brain barrier, to thereby slow or prevent the onset or progression of Alzheimer's disease.

In some embodiments, the patient is undergoing treatment with at least one additional agent for treatment of Alzheimer's disease, which may be selected from acetylcholinesterase inhibitors (tacrine, rivastigmine, galantamine and donepezil) or memantine.

For treatment of a patient showing potential symptoms of Alzheimer's, particularly early stage disease, the peptide or pharmaceutical composition described herein can be administered from about 1 to about 5 times daily, such as from about 1 to about 3 times daily to slow the onset or progression of the disease. Early stage disease can often be observed as an increasing impairment of learning and memory, which eventually leads to a definitive diagnosis. In some, difficulties with language, executive functions, perception (agnosia), or execution of movements (apraxia) are more prominent than memory problems. Language problems are characterized by a shrinking vocabulary and decreased word fluency, leading to a general impoverishment of oral and written language.

In some embodiments, the peptide or pharmaceutical composition is administered to a patient having early stage Alzheimer's Disease or a patient at risk for developing Alzheimer's Disease (either genetically predisposed, or is positive for one or more biomarkers associated with AD), where the peptide therapy normalizes circulation in the brain to slow or prevent disease progression.

In some embodiments, the patient has a neuropathology associated with dysregulation of angiogenesis or vascular leakage, such as multiple sclerosis (MS) or Parkinson's Disease (PD). In some embodiments, the peptide or pharmaceutical composition is administered 1 to 3 times daily to prevent or delay disease progression or to ameliorate disease symptoms.

In other embodiments, the patient has or is at risk of a hemorrhagic fever or syndrome, which are caused by hemorrhagic viruses. The most notorious of these are the Ebola and the Marburg viruses. Bleeding also occurs in people with Dengue or Lassa fever. In Ebola this hemorrhagic syndrome occurs somewhat late in the disease, typically 24 to 48 hours before death. Cases with bleeding can be dramatic and may occur from the nose, mouth and other orifices of the body. The mechanisms leading to the bleeding are known in broad outline: the virus causes up-regulation of clotting factors which are produced by the liver, the increased number of clotting factors cause clots to form in small blood vessels, the supply of clotting factors produced by the liver is exhausted because the liver is under attack by the virus, the hyper-activated immune system increases production of inflammatory proteins that cause the blood vessels to start bleeding, the unavailability of clotting factors means that the bleeding cannot be stemmed. Many deaths occur even without bleeding but patients with bleeding have a very high mortality rate. Agents administered after symptoms first appear could stop bleeding from the microvasculature in patients who would otherwise progress to display hemorrhagic syndrome.

In some embodiments, the patient has Ebola virus or Marburg virus. For example, the patient may have early signs of hemorrhagic fever, such as fever and increased susceptibility to bleeding, and/or flushing of the face and chest, small red or purple spots (petechiae). Other signs and symptoms of hemorrhagic fever include malaise, muscle pain, headache, vomiting, and diarrhea. In some embodiments, the presence of Ebola virus or other hemorrhagic fever virus is confirmed in patient samples. In some embodiments, the patient is undergoing treatment with at least one anti-viral agent or anti-inflammatory or agent for treatment of the hemorrhagic fever, such as intravenous ribavirin. For treatment of a patient having or at risk of hemorrhagic fever, the peptide or pharmaceutical composition described herein can be administered from about 1 to about 5 times daily, such as from about 1 to about 3 times daily, to slow the progression of the disease.

In still other embodiments, the patient has or is at risk of cerebral malaria (CM). CM is one of the most lethal complications of Plasmodium falciparum malaria and accounts for a large fraction of the malaria-related deaths. The World Health Organization (WHO) defines CM as coma (incapacity to localize a painful stimulus or Blantyre coma score <2) persisting at least 1 hour after termination of a seizure or correction for hypo-glycemia in the presence of asexual P. falciparum parasitemia and without the presence of other causes of encephalopathy. Up to 75% of CM-related deaths occur within 24 hours of admission. Multimodal magnetic resonance techniques such as imaging, diffusion, perfusion, angiography, spectroscopy have shown that vascular damage including blood-brain barrier disruption and hemorrhages occur in CM. These effects are thought to be due to inflammatory processes. Penet et al. (J Neurosci. 2005 Aug. 10; 25(32):7352-8) have shown using a mouse model of CM that major edema formation as well as reduced brain perfusion occurs in CM and is accompanied by an ischemic metabolic profile with reduction of high-energy phosphates and elevated brain lactate. They also used angiography which provided compelling evidence for major hemodynamics dysfunction. Importantly they found that edema further worsens ischemia by compressing cerebral arteries subsequently leading to a collapse of the blood flow that ultimately is the cause of death. These findings demonstrate the coexistence of inflammatory and ischemic lesions and prove the major role of edema in the fatal outcome of experimental cerebral malaria. Agents that inhibit edema and/or ischemia in the brain could be used in combination with anti-malarial agents that directly target the parasite to improve treatment of these patients. In some embodiments, the patient receives an anti-malarial therapy selected from chloroquine, mefloquine, doxycycline, or the combination of atovaquone and proguanil hydrochloride (Malarone).

In these embodiments, the peptide maintains the blood brain barrier and vascular integrity in patients with cerebral malaria. For treatment of a patient having or at risk of cerebral malaria, the peptide or pharmaceutical composition described herein can be administered from about 1 to about 5 times daily, such as from about 1 to about 3 times daily, to slow the progression of the disease and/or prevent death.

In other aspects, the invention provides a method for treating cancer, or normalizing a tumor microenvironment, and in particular for improving immune checkpoint inhibitor therapy.

The method comprises administering an effective amount of a peptide having the amino acid sequence of any one of SEQ ID NO:1 to 6, or a derivative or combination thereof, to a cancer patient undergoing therapy with (or in preparation for therapy) an immune checkpoint inhibitor. Angiogenesis is a drug target for treating cancer. VEGF and its receptor VEGFR2 are important mediators of angiogenesis. Bevacizumab, an antibody that sequesters human VEGF, and other small molecule tyrosine kinase inhibitors that inhibit VEGFR2 have been developed for various types of cancer. In addition to its well-known pro-angiogenic activity, VEGF also functions as an immune suppressor by inhibiting the maturation of dendritic cells. Tumors are thought to produce VEGF both to attract neovasculature and to suppress the immune system by reducing the number of mature immune cells and modulating lymphocyte endothelial trafficking.

Tumors marshal the immune system to promote their own growth. Over the last few years many of the mechanisms by which tumors keep the immune system in check have been deciphered. Many types of tumor cells express surface molecules such as PD-L1 and CTLA-4 that interact with receptors on T-cells that invade the tumor to make them quiescent. These discoveries have allowed the development of so-called “checkpoint inhibitors” such as ipilimumab, tremelimumab, nivolumab, and pembrolizumab as cancer drugs. These drugs are antibodies that interrupt the binding of the tumor cells to the cytotoxic T cells thus freeing them from suppression and allowing them to kill the tumor cells.

At least one study combining bevacizumab and ipilimumab, the antibody that blocks CTLA-4, has been conducted in patients with advanced melanoma (Cancer Immunol Res. 2014 July; 2(7):632-42). In addition to the effect of blocking VEGF on inflammation; lymphocyte trafficking and immune regulation were also apparent. Based on these studies more clinical trials combining bevacizumab and other anti-angiogenic agents such as small molecule tyrosine kinase inhibitors with checkpoint inhibitors have been initiated.

Other targets for checkpoint inhibitors, for which the peptides and compositions of the present invention may work synergistically, include LAG-3, KIR, OX40L, IDO-1, and TIM-3.

Further, the specificity of the peptide (e.g., SEQ ID NOS 1 to 6, and derivatives) for α5β1 and αVβ3 integrins, as disclosed herein, suggests other medically important roles for the peptide in cancer therapy. The receptors have been identified as the α5β1 and αVβ3 integrins. Integrins function as co-receptors for many different growth factor receptors. The peptides described herein and derivatives thereof inhibit signaling from the vascular endothelial growth factor receptor (VEGFR2), the hepatocyte growth factor receptor (c-met), and insulin-like growth factor receptor among others. P07, for example, strongly inhibits VEGF induced neovascularization and leakage in mouse models of retinal and choroidal neovascularization and vascular leakage. In addition to promoting angiogenesis, VEGF also causes immunosuppression which is exploited by the tumor to dampen the immune response against it. Since P07 blocks signaling by VEGF, it could effectively act as an immune system booster and thus promote attack on the tumor by the immune system. These data suggest that P07 (as well as other peptides disclosed herein and derivatives) could work well in combination with checkpoint inhibitors. It would help to simultaneously inhibit angiogenesis and strengthen the immune response against tumors by n the peptide maintains the blood brain barrier and vascular integrity in patients wi, so that the checkpoint inhibitor could allow infiltrating cytotoxic T-cells to kill tumor cells.

In some embodiments, the immune checkpoint inhibitor is an anti-PD-1 antibody, an anti-PD-L1 antibody, or an anti-CTLA-4 antibody. While the methods can be useful against any cancer where immune checkpoint therapy is effective, in some embodiments the cancer is a sarcoma, carcinoma, or solid tumor cancer selected from germ line tumors, tumors of the central nervous system, breast cancer, prostate cancer, cervical cancer, uterine cancer, lung cancer, ovarian cancer, testicular cancer, thyroid cancer, astrocytoma, glioma, pancreatic cancer, stomach cancer, liver cancer, colon cancer, melanoma (including advanced melanoma), renal cancer, bladder cancer, esophageal cancer, cancer of the larynx, cancer of the parotid, cancer of the biliary tract, rectal cancer, endometrial cancer, squamous cell carcinomas, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, lymphomas, melanomas, non-rhabdomyosarcome soft tissue sarcomas, osteosarcomas, peripheral primative neuroectodermal tumors, retinoblastomas, rhabdomyosarcomas, and Wilms tumors. In some embodiments, the cancer is non-small cell lung cancer, melanoma, prostate cancer, metastatic renal cell cancer. Generally, the cancer is positive for PD-1, PD-L1 or CTLA-4, and the checkpoint inhibitor therapy is an agent that inhibits an interaction between PD-1 and PD-L1 or CTLA-4 and B7.

In various embodiments, the patient can have either early stage cancer (e.g., stage I or II), or be in later stages (stage III or stage IV). Stage I cancers are localized to one part of the body. Stage II cancers are locally advanced, as are Stage III cancers. Whether a cancer is designated as Stage II or Stage III can depend on the specific type of cancer. For example, stage II can indicate affected lymph nodes on only one side of the diaphragm, whereas stage III indicates affected lymph nodes above and below the diaphragm. The specific criteria for stages II and III therefore differ according to diagnosis. Stage IV cancers have often metastasized, or spread to other organs or throughout the body.

In some embodiments, the cancer is non-resectable. A non-resectable cancer is a malignancy which cannot be surgically removed, due either to the number of metastatic foci, or because it is in a surgical danger zone.

In some embodiments, the patient is non-responsive or only partially responsive to the immune checkpoint inhibitor alone. While the peptide or pharmaceutical composition may be administered with (or during) immune checkpoint inhibitor therapy, in some embodiments the patient is treated for one to four weeks with the peptide, followed by immune checkpoint inhibitor therapy.

In some embodiments, the peptide or pharmaceutical composition is administered to reduce microvascular leakage or vascular permeability or lymphangiogenesis associated with organ or tissue transplantation, and thereby reduce the incidence of acute or hyperacute rejection. For example, the peptide can be administered to recipients for skin graft, corneal allograft, kidney, lung, or heart transplantation, or other organ or tissue transplantation, while at risk for acute or hyperacute rejection. For example, the peptide may be administered at least once daily for from one to eight weeks, or from one to four weeks.

In the various embodiments described above, the peptide can be administered in a variety of forms depending on the desired route and/or dose.

The peptide can be delivered as a pharmaceutically acceptable salt, and may include any number of carriers known in the art. The term “pharmaceutically acceptable salt” includes salts that are prepared with relatively nontoxic acids or bases. As used herein, “pharmaceutically acceptable carrier” is intended to include, but is not limited to, water, saline, dextrose solutions, human serum albumin, liposomes, hydrogels, microparticles and nanoparticles.

Depending on the specific conditions being treated, the peptide agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

While the form and/or route of administration can vary, in some embodiments the peptide or pharmaceutical composition is administered parenterally (e.g., by subcutaneous, intravenous, or intramuscular administration), or in some embodiments is administered directly to the lungs. Local administration to the lungs can be achieved using a variety of formulation strategies including pharmaceutical aerosols, which may be solution aerosols or powder aerosols. Powder formulations typically comprise small particles. Suitable particles can be prepared using any means known in the art, for example, by grinding in an airjet mill, ball mill or vibrator mill, sieving, microprecipitation, spray-drying, lyophilization or controlled crystallization. Typically, particles will be about 10 microns or less in diameter. Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. Alternatively, solution aerosols may be prepared using any means known to those of skill in the art, for example, an aerosol vial provided with a valve adapted to deliver a metered dose of the composition. Where the inhalable form of the active ingredient is a nebulizable aqueous, organic or aqueous/organic dispersion, the inhalation device may be a nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer which allows smaller nebulized volumes, e.g. 10 μl to 100 μl.

For injection, the agents of the disclosure may be formulated and diluted in aqueous solutions, such as in physiologically compatible buffers such as Hank's solution, Ringer's solution, or physiological saline buffer.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In some embodiments, the peptide is formulated with a polymeric nanoparticle or microparticle carrier. For example, in some embodiments, the microparticle or nanoparticle comprises a material having one or more degradable linkages, such as an ester linkage, a disulfide linkage, an amide linkage, an anhydride linkage, and a linkage susceptible to enzymatic degradation. In particular embodiments, the microparticle or nanoparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In other embodiments, nondegradable polymers that are used in the art, such as polystyrene, are blended with a degradable polymer or polymers from above to create a copolymer system. Accordingly, in some embodiments, a nondegradable polymer is blended with the biodegradable polymer.

In some embodiments, the invention provides a nanoparticle comprising PLGA-PEG copolymers and a conjugated peptide targeting integrins. The conjugated peptide can be a peptide of any one or more of SEQ ID NO:1 to 31, or derivatives thereof. For example, N07 is a designation used herein for a peptide-conjugated nanoparticle based on P07 that has anti-angiogenic and anti-tumorigenic properties. N07 has anti-angiogenic and anti-tumorigenic activity in vitro, specific binding to the integrin αVβ3 complex, and the ability to carry encapsulated drug cargo.

In some embodiments, the nanoparticles contain an additional drug or targeting agent conjugated to the surface. For example, the nanoparticles may be made from PLGA-PEG-X and PLGA-PEG-Y polymers, where X is said peptide and Y is another drug or targeting agent. The targeting agent may be a tissue selective targeting agent, or may be selective for cancer cells. Nanoparticles in these embodiments (having conjugated peptide, and optionally an additional targeting agent) may be used in a treatment of cancer, including solid tumors as described above, and including glioblastoma or breast cancer (including triple negative breast cancer).

Other target binding agents may be used, in addition or alternatively (including alternative integrin binding moieties), and these include antibodies and antigen-binding portions thereof. The various formats for target binding include a single-domain antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a peptide aptamer, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, a peptide mimetic molecule, or a synthetic molecule, or as described in US Patent Nos. or Patent Publication Nos. U.S. Pat. No. 7,417,130, US 2004/132094, U.S. Pat. No. 5,831,012, US 2004/023334, U.S. Pat. No. 7,250,297, U.S. Pat. No. 6,818,418, US 2004/209243, U.S. Pat. No. 7,838,629, U.S. Pat. No. 7,186,524, U.S. Pat. No. 6,004,746, U.S. Pat. No. 5,475,096, US 2004/146938, US 2004/157209, U.S. Pat. No. 6,994,982, U.S. Pat. No. 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the nanoparticle is synthesized from poly(lactic-co-glycolic acid) polyethylene glycol (PLGA-PEG) block copolymers of tunable size which are covalently linked to the peptide (e.g., comprising the sequence of any one of SEQ ID NOS:1 to 6, or derivative thereof), or other binding agent as described above. A mix of conjugated and unconjugated polymers in any ratio can be used to create nanoparticles with the desired density of targeting agent on the surface. A description of the tunable characteristics of the particle can be found in Table 1.

In some embodiments, the peptide conjugated to the particle has the amino acid sequence of SEQ ID NO:1, SEQ ID NO:2, SEQ ID NO:3, SEQ ID NO:4, SEQ ID NO:5, or SEQ ID NO:6, or derivative thereof (e.g., including a peptide of SEQ ID NOS:7-31) as described. The nanoparticles in some embodiments are formed from PLGA-PEG-peptide conjugates, or in other embodiments, peptide is conjugated to pre-formed particles.

As used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 50 to about 100 nm. In some embodiments, the nanoparticle has a diameter of about 70 to 100 nm.

In some embodiments, the particle is a microparticle. The term “microparticle” includes particles having at least one dimension in the range of at least about one micrometer (μm). The term “particle” as used herein is meant to include nanoparticles and microparticles.

The particles may be designed to provide desired pharmacodynamic advantages, including circulating properties, biodistribution, and degradation kinetics. Such parameters include size, surface charge, polymer composition, ligand conjugation chemistry, peptide conjugation density, among others. For example, in some embodiments, the particles have a PLGA polymer core, and a hydrophilic shell formed by the PEG portion of PLGA-PEG co-polymers, wherein a portion of the PLGA-PEG polymers have a terminal attachment of the peptide. The hydrophilic shell may further comprise ester-endcapped PLGA-PEG polymers that are inert with respect to functional groups, such as PLGA-PEG-MeOH polymers. In some embodiments, some or all of the unconjugated polymers have other terminal groups (such as carboxy) to provide fine tuning of the surface properties.

Peptides described herein can be chemically conjugated to the particles using any available process. Functional groups for peptide conjugation include PEG-COOH, PEG-NH₂, PEG-SH. See, e.g., Hermanson, BIOCONJUGATE TECHNIQUES, Academic Press, New York, 1996. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, peptides can be conjugated through the use of a small molecule-coupling reagent. Non-limiting examples of coupling reagents include carbodiimides, maleimides, N-hydroxysuccinimide esters, bischloroethylamines, bifunctional aldehydes such as glutaraldehyde, anhydrides and the like.

In an exemplary embodiment, the nanoparticles have a core (PLGA) that can be tuned for a specific biodegradation rate in vivo (by adjusting the LA:GA ratio and/or molecular weight of the PLGA polymer). In some embodiments, the PLGA is based on a LA:GA ratio of from 20:1 to 1:20, including compositions of L/G of: 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. PLGA degrades by hydrolysis of its ester linkages. The time required for degradation of PLGA is related to the ratio of monomers: the higher the content of glycolide units, the lower the time required for degradation as compared to predominantly lactide units. In addition, polymers that are end-capped with esters (as opposed to the free carboxylic acid) have longer degradation half-lives.

In some embodiments, the PLGA polymers for fabricating nanoparticles have a molecular weight in the range of about 10K to about 70K, such as about 20K, about 25K, about 30K, about 40K, about 50K, about 60K, or about 70K, to provide tunable particle size. In some embodiments, the PLGA polymers used for fabricating microparticles have a molecular weight in the range of about 20K to about 200K, such as from 100K to about 200K. The PEG portion of the polymer is generally in the range of 2K to 5K. In various embodiments, the ratio of PLGA-PEG-peptide and unconjugated PLGA-PEG ranges from about 1:20 to about 20:1, such as from about 1:15 to about 15:1, or about 1:10 to about 10:1, or about 1:5 to about 5:1, or about 1:2 to about 2:1. In some embodiments, the ratio of PLGA-PEG-peptide and unconjugated copolymers is about 1:1. In some embodiments, at least 50% of the polymers have conjugated peptide. In some embodiments, the nanoparticle has a size (average diameter) within the range of about 50 to about 200 nm, or within the range of about 50 to about 100 nm. In some embodiments, the nanoparticle has a zeta potential in deionized water within the range of about −5 mV to about −40 mV, and in some embodiments, from about −10 mV to about −30 mV (e.g., about −20, about −25, or about −30 mV).

In some embodiments, the nanoparticle further comprises an encapsulated active agent, which may be an active agent disclosed herein for treatment of conditions characterized by microvascular leakage, including flu, Alzheimer's Disease, hemorrhagic fever, cerebral malaria, cancer, prevention of acute rejection, and others described herein. In these embodiments, the nanoparticle provides a sustained release of the active agent. For example, in some embodiments, the active agent is a chemotherapeutic agent, such as one or more of: aminoglutethimide, amsacrine, anastrozole, asparaginase, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rapamycin, rituximab, streptozocin, suramin, tacrolimus, tamoxifen, temozolomide, teniposide, testosterone, thioguanine, thiotepa, titanocene dichloride, topotecan, trastuzumab, tretinoin, vinblastine, vincristine, vindesine, and vinorelbine.

While the nanoparticle is substantially spherical in some embodiments, the nanoparticle may optionally be non-spherical.

There are various physical and chemical properties that can affect how a material interacts with a biological system. In the case of microparticle and nanoparticle based materials, the choice of material, the size distribution, and the shape distribution of the particles are all critical parameters affecting the particles' activity. It has been previously shown that both the size and shape of a particle can affect the way the particle interacts with various cells of the body. For example, the shape of the particle can affect how well various cell types can uptake the particle, where an ellipsoidal particle is usually more difficult for a cell to uptake than a spherical particle. Stretching the shape of the particles can therefore reduce unwanted uptake of particles, such as by the immune system cells, thereby extending the half-life of the particles in the body. The size of the particle also affects the ability of cells to uptake and interact with the particles. Optimization of the activity of a particle based system can therefore be achieved by tuning the size and shape distribution of the particles.

In some embodiments, the dimensions of the nanoparticle and/or process for stretching the particles in as disclosed in WO 2013/086500, which is hereby incorporated by reference in its entirety.

In particular embodiments, the three-dimensional microparticle or nanoparticle comprises a prolate ellipsoid, wherein the dimension (a) along the x-axis is greater than the dimension (b) along the y-axis, and wherein the dimension (b) along the y-axis is substantially equal to the dimension (c) along the z-axis, such that the prolate ellipsoid can be described by the equation a>b=c. In other embodiments, the ellipsoid is a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c. In yet other embodiments, the ellipsoid is an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c. The presently disclosed asymmetrical particles, however, do not include embodiments in which a=b=c.

In still other embodiments, the microparticle or nanoparticle has an aspect ratio ranging from about 1.1 to about 5. In other embodiments, the aspect ratio has a range from about 5 to about 10. In some embodiments, the aspect ratio has a range from about 1.5 to about 3.5.

In some embodiments, the particle is a microparticle that encapsulates a drug cargo (such as a peptide described herein, and/or other agent). The particle may or may not contain peptide conjugated to the surface in these embodiments. In these embodiments, the particle can provide a long acting drug depot, to provide a sustained release of peptide. Exemplary particle formats include those described in WO 2014/197892, which is hereby incorporated by reference. In some embodiments, particles do not incorporate poly(beta-amino ester) (PBAE), and thus the polymers consist essentially of PLGA-PEG block co-polymers. These particles can be used for intraocular injection, for example, as a treatment for macular degeneration (e.g., wet or dry age-related macular degeneration) or diabetic macular edema. In some embodiments, the cargo allows for a combination of active agents to be delivered to desired site. In some embodiments, the nanoparticle is administered for the treatment of cancer. In these or other embodiments, the particle has a size (average diameter) in the range of 1 μm to 500 of μm, such as in the range of about 1 μm to about 250 μm. The particles can be injected from about once daily to about once every six months, or about weekly or about monthly, depending on the duration of the sustained peptide or drug release.

In other aspects, the invention provides a method for identifying expression of integrins on one or more cells. For example, in some embodiments the method comprises contacting the nanoparticle or microparticle having the peptide of SEQ ID NO:1, SEQ ID NO:2, SEQ ID NO:3, SEQ ID NO:4, SEQ ID NO:5, or SEQ ID NO:6 (or derivative of any one of SEQ ID NOS:1-6 as described, including the peptides of SEQ ID NOS:7 to 31) conjugated to the surface (as described above) with one or more cells, and visualizing or detecting binding of the nanoparticle to cells. In some embodiments, the nanoparticles further comprise a detectable label, such as a fluorescent, luminescent, enzymatic, or radioactive label, which can be conjugated to a portion of the PLGA-PEG polymers, encapsulated in the nanoparticles, or indirectly bound through other moieties. The cells may be in solution or in culture in some embodiments. For in vitro applications, binding can be determined by direct visualization of bound particles, flow cytometry, or by pull down of cells from solution. In some embodiments, magnetic particles, as opposed to polymeric particles, are used to allow a convenient method for separating cells expressing the targeted integrins.

In still other embodiments, the nanoparticles are administered to a patient, and integrin over-expressing vasculature is imaged, for example, in the vicinity of a tumor.

### EXAMPLES

### Example 1: P07 Inhibits Signaling of VEGF, HGF and IGF

The targets of P07 were identified to be the α5β1 and αVβ3 integrins. Integrins function as co-receptors for many growth factor receptors such as VEGFR, hepatocyte growth factor receptor (HGFR or c-met), insulin-like growth factor receptor (IGFR), and platelet derived growth factor receptor (PDGFR). Consistent with this mechanism, P07 was found to inhibit signaling from these receptors (FIG. 1).

These receptors and others are involved in angiogenesis and microvascular permeability. This multi-factorial inhibition makes it likely that diseases involving multiple mechanisms can be effectively treated with P07 and its derivatives.

### Example 2: P07 Inhibits Neovascularization in Multiple Ocular Models

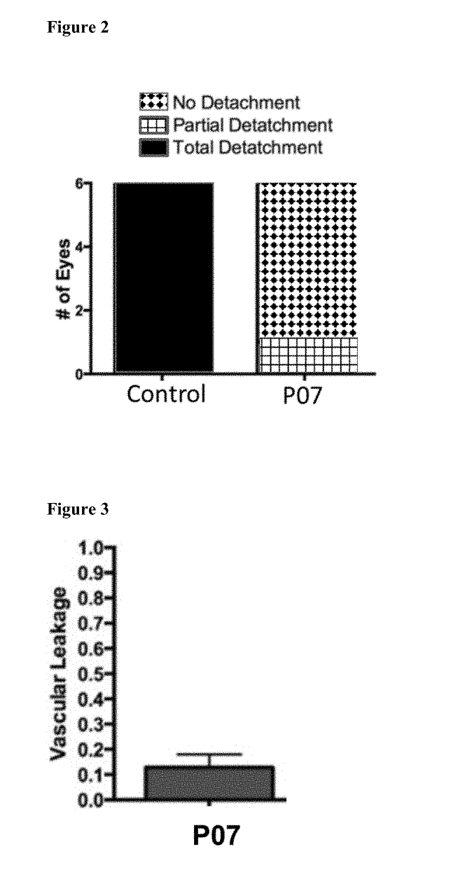

P07 was found to inhibit vascular leakage in a transgenic mouse over-expressing the human form of vascular endothelial growth factor (VEGF) in the retina. The vascular leakage in this model is so severe that the blood that pools behind the retina causes retinal detachment. In this model, P07 almost completely blocked retinal detachment (FIG. 2).

P07 was also tested in a model of edema in the rabbit eye. In this model human VEGF was injected directly into the eye causing the local vasculature to be leaky. The extent of this leak was assessed by measuring the amount of fluorescence in the eye resulting from the leakage of sodium fluorescein administered intravenously 3 days after VEGF injection. When P07 was present at about 50 μg in the eye prior to VEGF injection, the vascular leakage was dramatically inhibited (FIG. 3). These results suggest that P07 is a potent anti-edemic agent in vivo.

### Example 3: P07 Inhibits Cancer Tissue Growth

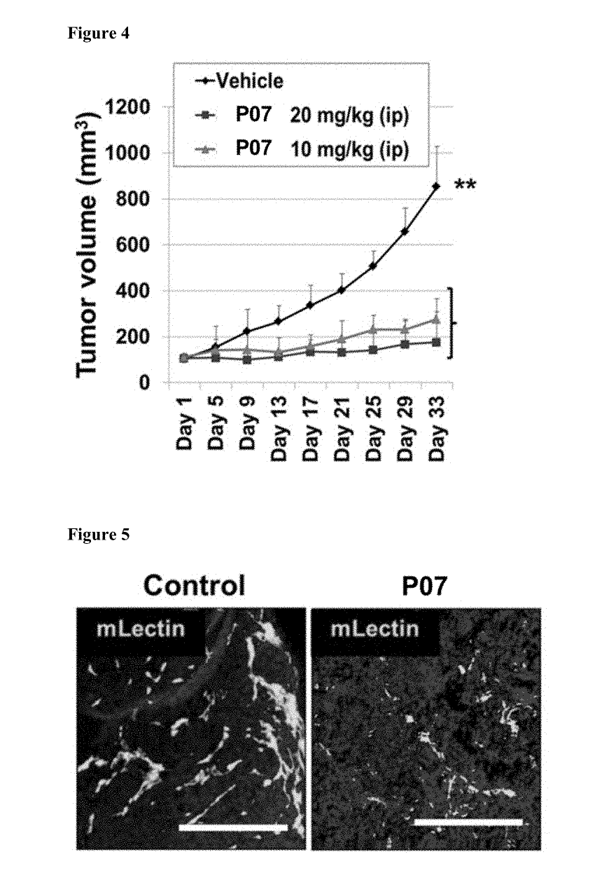

P07 inhibits the growth of orthotopic triple-negative breast cancer (TNBC) xenografts (FIG. 4) and small cell lung cancer (SCLC) and glioblastoma xenografts (not shown). The responding tumors have dramatically diminished vasculature (FIG. 5).

These results indicate that P07 may have synergistic effects when combined with immune checkpoint inhibitors. P07 and an immune checkpoint inhibitor can simultaneously inhibit angiogenesis and strengthen the immune response against tumors by allowing dendritic cell maturation and more robust lymphocyte endothelial trafficking and the checkpoint inhibitor would allow infiltrating cytotoxic T-cells to kill tumor cells.

### Example 4: The Properties of P07-Conjugated Nanoparticle (N07)

N07 is a peptide-conjugated nanoparticle that has anti-angiogenic and anti-tumorigenic properties. N07 specifically binds to the integrin αVβ3 complex, and has the ability to carry encapsulated drug cargo.

N07 was synthesized from poly(lactic-co-glycolic acid) polyethylene glycol (PLGA-PEG) block copolymers of tunable size which are covalently conjugated with N07. A mix of conjugated and unconjugated polymers in any ratio can be used to create nanoparticles with the desired density of P07 on the surface. A description of the tunable characteristics of the particle is shown in Table 1.

TABLE 1

| Tunable Parameter | Applicable Range |
|---|---|
| Molecular weight of PLGA | 10 kDa-70 kDa |
| Molecular weight of PEG | 2 kDa-5 kDa |
| Percent of polymers conjugated to peptide | 0-100% |

N07 was synthesized from the P07 peptide and PLGA-PEG block copolymers. P07 was produced by solid state synthesis at New England Peptide and its purity assessed by HPLC/MS. The peptide has an amine at the N-terminus and an amide at the C-terminus. PLGA-PEG block copolymers were purchased from PolySciTech® and purity and molecular weight was assessed by gel permeation chromatography (GPC) and Fourier transform infrared spectroscopy (FTIR).

For conjugation, P07 was dissolved at 100 mg/ml in DMSO and added to 170 mg/ml NHS-functionalized PLGA-PEG (PLGA-PEG-NHS) in DMF. 40 molar excess of diisopropylethylamine (DIPEA) was added to the mixture and stirred overnight at room temperature. The mixture was then added dropwise to a cold mixture of ether and methanol and spun down at 22,000×g. The pellet was then repeatedly washed with methanol and spun down to remove unreacted peptide. The supernatant was discarded, and the pellet was left to dry under vacuum for several hours to yield solid PLGA-PEG-SP2043. Other cargo, such as drugs and dyes, can also be conjugated to PLGA-PEG via a similar process.

A mixture of PLGA-PEG-P07 and PLGA-PEG was dissolved in DMF at 10 mg/ml and added dropwise to deionized water under magnetic stirring to form nanoparticles in a process called nanoprecipitation. Desired cargo was added to the DMF mixture prior to nanoprecipitation, which resulted in a nanoparticle with loaded cargo in the hydrophobic PLGA core. After several hours of stirring, the particles were filtered and concentrated with Ultra Centrifugation Columns (EMD Millipore, UFC810096).

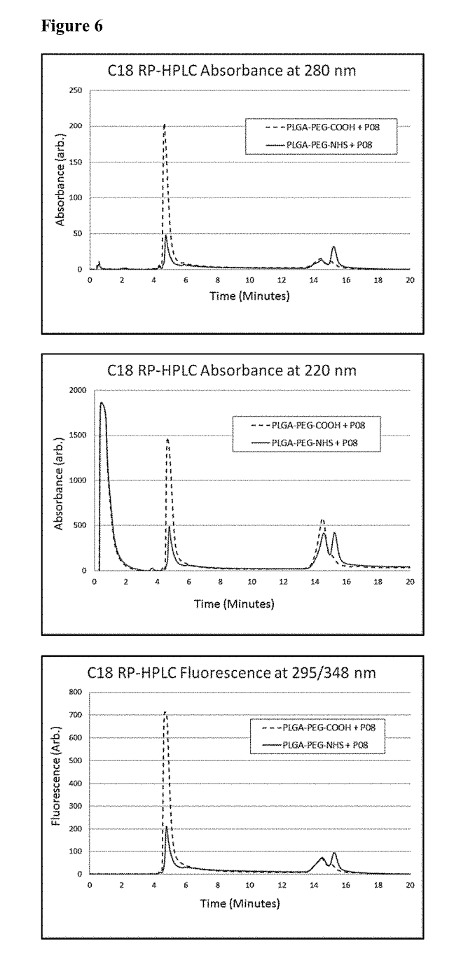

HPLC was used to assess the efficiency of the conjugation of PLGA-PEG-NHS to P07. In some cases, the c-terminal F was replaced with W to allow reading of absorbance and fluorescence from the W residue. PLGA-PEG-P07 from the reaction mixture prior to precipitation in ether and methanol was diluted in DMSO and run through an Agilent Poroshell 300 column in a water-acetonitrile mobile phase. Peptide was detected via absorbance at 220 nm (peptide backbone) and 280 nm (tryptophan, W), and via fluorescence at 295/348 nm excitation/emission (tryptophan). The amount of unreacted P07 was determined via integration of the free P07 peak. The PLGA-PEG-P07 reaction mixture was compared to a control reaction mixture of PLGA-PEG-COOH or PLGA-mPEG and P07, neither of which will undergo a reaction to form PLGA-PEG-P07. Reacted P07 would not contribute to the free P07 peak, so a reduction in the P07 peak relative to the control reaction indicates conjugation to the PLGA-PEG-NHS copolymer. Results were compared to a standard curve of free peptide to ensure all integration values fell within a linear range of peptide concentration. The results are shown in Table 2 and FIG. 6.

TABLE 2

| Signal for Quantification | Conjugation Efficiency |
|---|---|
| Fluorescence (295/348 nm) | 0.869 |
| Absorbance (280 nm) | 0.920 |
| Absorbance (220 nm) | 0.854 |

LavaPep™ Characterization: The LavaPep™ peptide quantification kit (Gel Company, LP022010) was used to directly detect peptide on the surface of N07. Particles at 3-5 mg/ml in ultrapure water were incubated for 1 hour in the dark with the LavaPep working solution in 96-well plates. The epicocconone dye in the LavaPep working solution interacted with the Arg residues on the peptide to become highly fluorescent. Fluorescence was read at 530/590 nm on a Biotek HT Synergy plate reader. The signal from the nanoparticles was compared to a standard dilution curve of known amounts of free peptide. The quantification results are shown in Table 3, demonstrating that SP2043 was conjugated efficiently to PLGA-PEG-NHS copolymers and incorporated into nanoparticles.

TABLE 3

| Surface | | | | |
|---|---|---|---|---|
| percentage | Average | Corresponding | Peptide Standard | Conjugation |
| SP2043 | Signal | Peptide (μg) | Deviation (μg) | Efficiency |
| 100 | 1992 | 14.80 | 0.538 | 0.889 |
| 50 | 1432 | 7.01 | 0.249 | 0.842 |
| 0 | 0 | 0 | — | — |

Size Characterization:

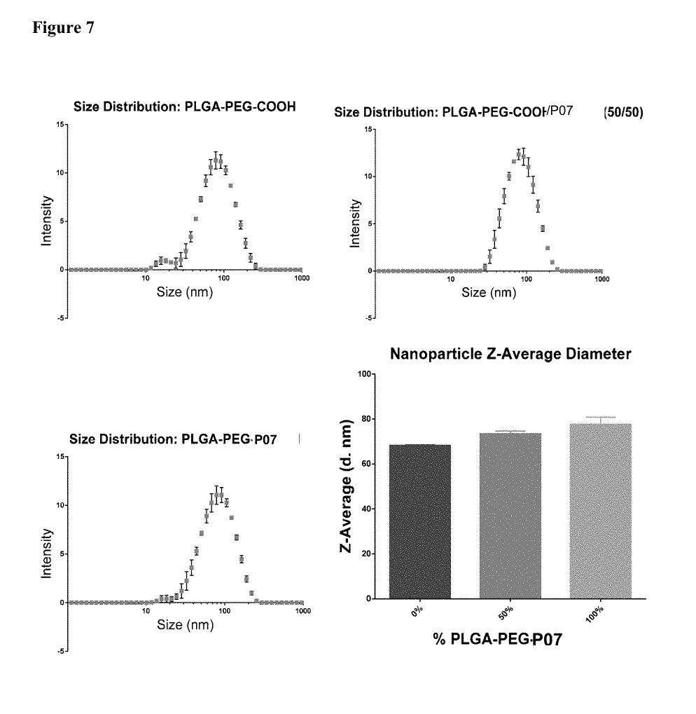

N07 was suspended in water at 1 mg/ml and analyzed using a Malvern Zetasizer Nano ZS90. Z-average diameter and intensity-based size distribution were measured and recorded. The results are shown in Table 4 and FIG. 7, demonstrating that N07 exhibits a Z-average diameter of approximately 70-80 nm and slight size increase was seen in samples with conjugated P07 on the nanoparticle.

TABLE 4

| Sample | Z-Average (d · nm) | PDI |
|---|---|---|
| PLGA-PEG-COOH | 68.24 ± 0.26 | 0.23 |
| PLGA-PEG-COOH/P07 (50/50) | 73.39 ± 1.03 | 0.18 |
| PLGA-PEG-P07 | 77.59 ± 2.65 | 0.29 |

Zeta-Potential:

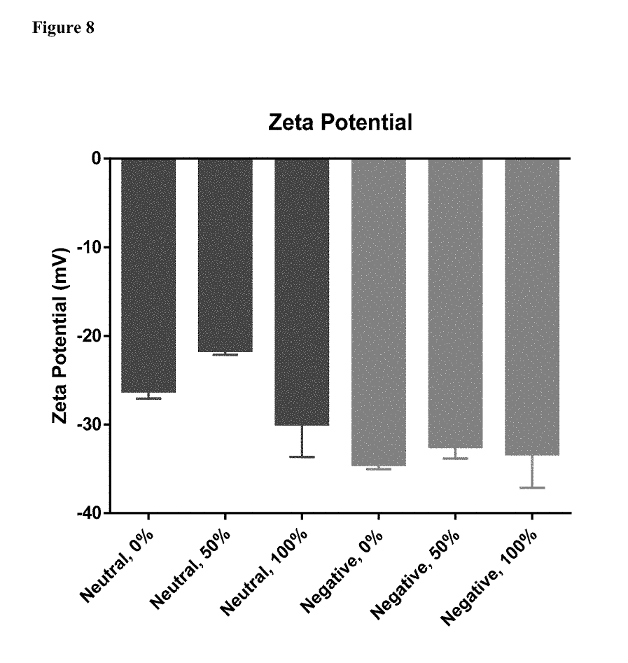

N07 was suspended in ultrapure water at 1 mg/ml and analyzed using a Malvern Zetasizer Nano ZS90. Surface zeta potential was measured and recorded. The results are shown in FIG. 8, demonstrating that N07 exhibits a negative zeta potential in deionized water, which is very slightly tunable around 25 mV using different end groups on PEG. In FIG. 8, neutral is methoxy-terminated PEG and negative is carboxy-terminated PEG.

### Example 5: Binding of PLGA-PEG-P07 to Integrin αvβ3 and Integrin α5β1 Targets

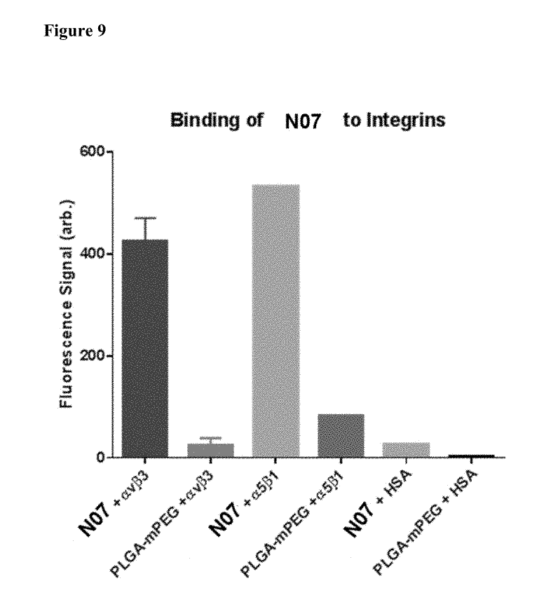

Particles were prepared as described above, made either completely of PLGA-PEG-P07 or PLGA-mPEG. Integrin αvβ3, integrin α5β1, and human serum albumin were labeled with an Alexafluor 488 TFP-ester according to the manufacturer's instructions. Particles were incubated at room temperature with the integrin in PBS overnight alongside a control sample with integrins or HSA in PBS without particles. Particles were then separated from free integrins or HSA protein via SEC centrifugal spin columns using Sephacryl S-500 HR media. After separation, fluorescence signal was measured on a Biotek Synergy HT microplate reader to assess the amount of integrin brought through the SEC media by the particles. The results are shown in FIG. 9.

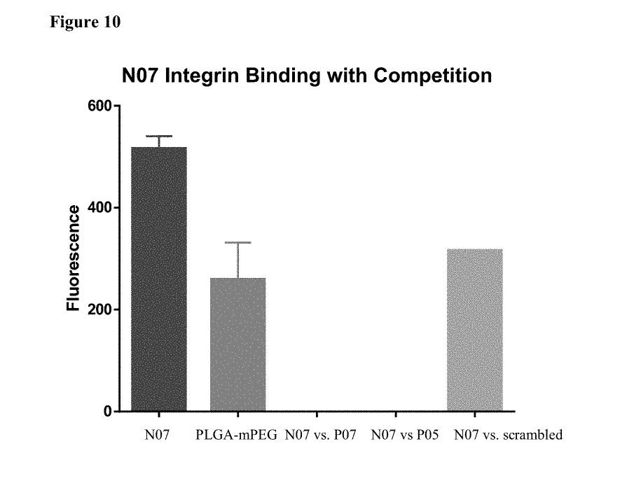

The above protocol was repeated for binding to integrin αvβ3, but with the addition of competition samples. In brief, various peptides (P07 and a partial scramble of the sequence of P07 that has been shown to be inactive) were added to the solution of targeted particles and Alexafluor 488-labeled free integrins at 100 times excess and incubated at room temperature overnight. The solutions were separated via SEC centrifugal spin columns using Sephacryl S-500 HR media. As before, fluorescence was used to assess the amount of integrin brought through the SEC media by the particles. The results are shown in FIG. 10.

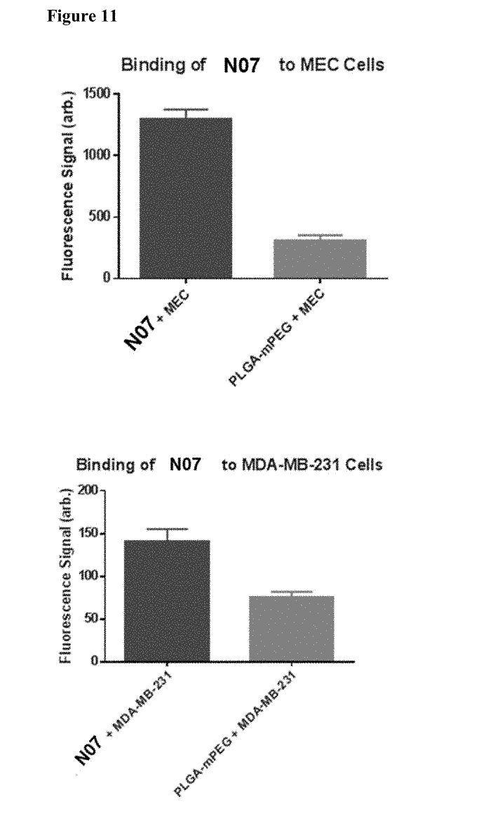

Nanoparticles were tested for binding to MDA-MB-231 and microvascular endothelial cells (MEC) Cells. Particles were prepared as above with the addition of 1% TAMRA dye by weight. After nanoprecipitation, the particles were concentrated with Amicon ultracentrifugation filters (MWCO 100,000) and filtered with SEC centrifugal spin columns using Sephacryl S-500 HR media to remove free TAMRA dye or other free polymer or peptide materials. Cells were then incubated with nanoparticles at 100,000 cells/ml and 1 mg/ml particle. Nanoparticles were made from either targeted PLGA-PEG-P07 polymers or untargeted PLGA-mPEG polymers. After 1 hour incubation at 37° C., the cells were spun down in the centrifuge and the supernatant was removed. Cells were resuspended in PBS and the resulting signal was on a Biotek Synergy HT microplate reader to assess the amount of fluorescent particle brought down with the cells. The results are shown in FIG. 11.

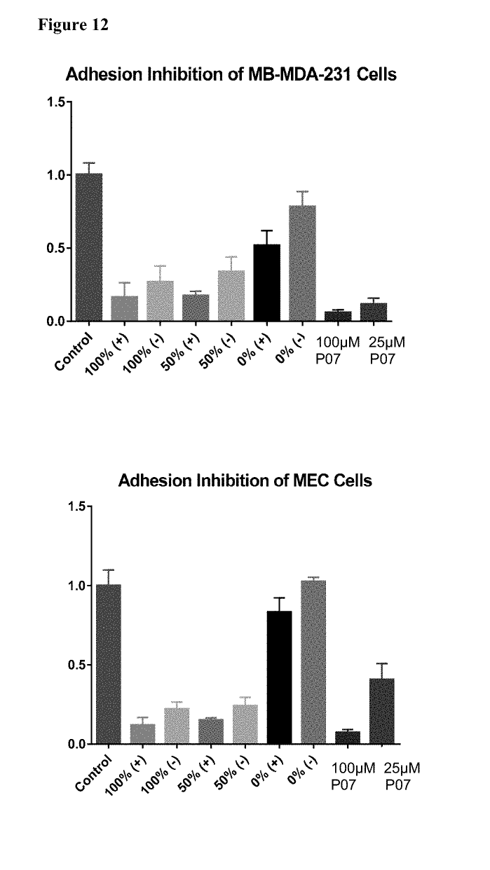

Particles were tested for their ability to inhibit adhesion of MB-MDA-231 cells and microvascular endothelial cells (MEC). Prior to use in in vitro assays, particles were transferred from ultrapure water to appropriate media using ultracentrifugation columns, concentrated to 10 mg/ml in media, and added to 96-well plates. Media alone and media with 100 M and 25 M AXT201 (a known inhibitor of adhesion) were added to plates as positive and negative controls. MDA-MB-231 or MEC cells were added at 20,000 cells/well to the particles, peptides, and media. The 96-well plate was incubated for approximately 2 hours at 37° C. and 5% CO₂. Wells were then washed twice with DPBS with Ca′ and Mg′ and then filled with media containing 4 μg/ml Calcein AM dye. Plates were then incubated for 30 minutes and washed again with DPBS with Ca²⁺ and Mg²⁺. Fluorescence was then read on a Biotek Synergy HT at 485/528 nm excitation/emission to quantify the number of cells adhered to the surface of the well. The results are shown in FIG. 12, demonstrating that N07 has anti-adhesion activity against MDA-MD-231 tumor cells and MEC cells. In FIG. 12, the % refers to amount of PLGA-PEG molecules that have conjugated P07. “+” or “−” refers to the presence or absence of encapsulated P07.

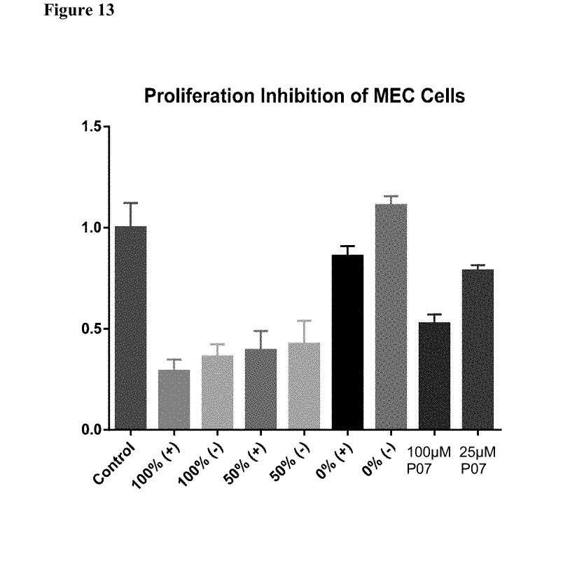

Particles were tested for inhibition of MEC proliferation. Colorimetric based proliferation assay using the MTT Vybrant Assay Kit were carried out on MEC cells. 2000 cells/well were plated in 96-well plates in phenol red-free ECM-2MV media and allowed to adhere over 18-20 hours. Original media with no particles was replaced with N07 particles suspended in media at 5 mg/ml or AXT201 peptide in media or media alone. After four days, media was replaced with 100 ul MTT reagent as per the manufacturer's recommendations. After four hours, 100 ul of SDS solution was added to each well and incubated at 37° C. for another four hours. Absorbance was read at 570 nm on a Biotek Synergy HT plate reader to capture the change from MTT to formazan by mitochondrial reductase in the living cells. The results are shown in FIG. 13, demonstrating that N07 has anti-proliferation activity against MEC cells. In FIG. 13, the % refers to amount of PLGA-PEG molecules that have conjugated P07. “+” or “−” refers to the presence or absence of encapsulated P07.

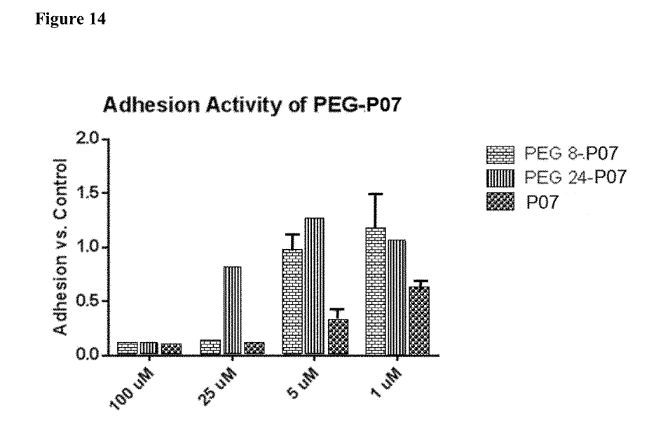

The ability of PEG-P07 conjugates to inhibit adhesion was tested. NHS-functionalized PEG 8 and PEG 24 were dissolved in DMF at approximately 150 mg/ml. Peptide was added at a 1:1 molar ratio in DMSO at 100 mg/ml along with a 40 fold molar excess of DIPEA. The mixture was precipitated in cold ether and methanol and washed several times to remove DIPEA, solvents, and free PEG. The resulting mixture was then used in an adhesion assay using MDA-MB-231 cells as described above alongside positive and negative controls. The results are shown in FIG. 14, demonstrating the adhesion activity of PEG-P07.

### Example 6: Stretching the Peptide-Conjugated Particles

The size and shape of particles were manipulated for the optimization of the activity of the particles. The particles were made and stretched successfully. The peptides remain stable throughout the process, with peptide loading remaining the same before and after the stretching protocol.

Microparticle Formation:

Poly(lactide-co-glycolide), PLGA, was first dissolved into dichloromethane, DCM, at 20 mg/mL in a test tube and vortexed to fully dissolve. Peptide stock of P07 in dimethylsulfoxide, DMSO (20 mg/mL) was micropipetted to the PLGA/DCM solution. The initial mass ratio of peptide to PLGA can vary; such as 1:50 and 1:20 peptide:PLGA. For blank microparticle, equivalent volume of DMSO only was pipetted. The mixture was sonicated with the test tube on ice. Sonication was performed with an amplitude setting of ‘30’, which equals approximately 5-10 W, for 20 seconds. This primary emulsion was immediately poured into 50 mL of 1% poly(vinyl alcohol), PVA, solution and homogenized at 3.6-3.8 krpm for 1 minute. The full volume was then transferred to 100 mL of 0.5% PVA solution and stirred in a chemical hood for about 3.5 hours. Three wash steps were then performed. For each wash step, the microparticle solution was centrifuged at 4° C., 4 krpm, for 5 minutes, and then the supernatant was removed. Subsequently, 40 mL of refrigerated Milli-Q water was added, the microparticle pellet was resuspended and the washing steps were repeated. After the last centrifugation step, 5 mL of water was added to resuspend the sample. Samples were snap frozen in liquid nitrogen and immediately placed in a lyophilizer. Following lyophilization, all microparticles were stored at −20° C.

Nanoparticle Formation:

PLGA was first dissolved into DCM, at desired concentration (usually 20 mg/mL or 40 mg/mL), in a test tube and vortexed to fully dissolve. Peptide stock, such as P07, in DMSO (20 mg/mL) was micropipetted to the PLGA/DCM solution. The mass ratio of peptide to PLGA can vary. An exemplary formulation is 1:50 peptide:PLGA. For blank nanoparticle, an equivalent volume of DMSO only was pipetted. The mixture was sonicated with the test tube on ice. Sonication (Misonix) was performed with an amplitude setting of ‘30’, which equals approximately 5-10 W, for 20 seconds. This primary emulsion was immediately poured into 50 mL of 1% PVA solution and sonicated at an amplitude setting of anywhere from ‘30’ to ‘100’ for 2 minutes on ice. The full volume was then transferred to 100 mL of 0.5% PVA solution and stirred in a chemical hood for ˜3.5 hours. Three wash steps were then performed. For each wash step, the nanoparticle solution was centrifuged at 4° C., 17 krpm, for 10 minutes, and then the supernatant was removed. Subsequently, 30 mL of refrigerated Milli-Q water was added, the nanoparticle pellet was resuspended and the washing steps were repeated. After the last centrifugation step, 5 mL of water was added to resuspend the sample. Samples were snap frozen in liquid nitrogen and immediately placed in a lyophilizer. Following lyophilization, all nanoparticles were stored at −20° C.

Microparticle Stretching:

Lyophilized PLGA particles were dissolved in a 10% PVA/2% glycerol solution at a concentration of 2.5 mg/mL and 10 mL of this solution was deposited into rectangular petri dishes to dry overnight. The resulting film was cut to size and loaded in between two aluminum mounts and heated up to 90° C. The film length was measured and the film was stretched slowly to produce the desired fold of stretch (e.g. 2 fold stretched ellipsoidal particles) using custom made stretching device. The film was then allowed to cool down to room temperature and was removed from the aluminum blocks. The PVA film was dissolved in water and the resulting particle suspension was washed 3×. The particles were lyophilized prior to use.

SEM and TEM Characterization:

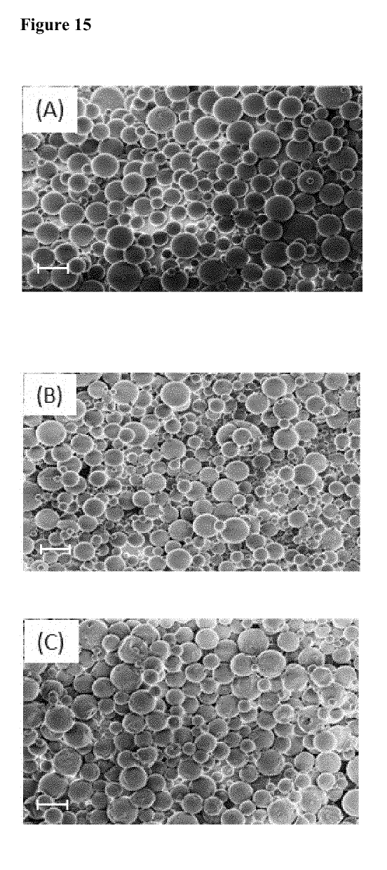

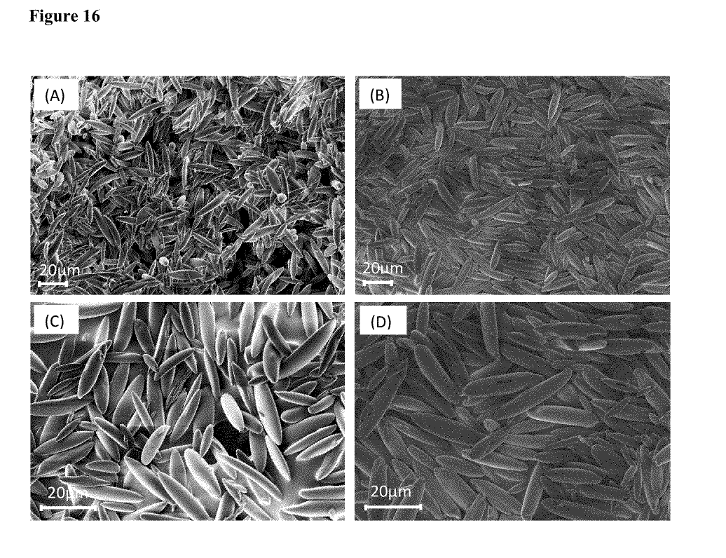

For scanning electron microscope (SEM), Lyophilized particles were placed on carbon tape (Electron Microscopy Sciences, Hatfield, Pa.) placed on aluminum mounts. Samples were sputtered with gold-palladium, and SEM imaging was performed with a LEO/Zeiss FESEM at the JHU School of Medicine MicFac. Sizing of microparticle samples was performed with ImageJ analysis of SEM images. The results are shown in FIG. 15. Stretched particles are shown in FIG. 16.

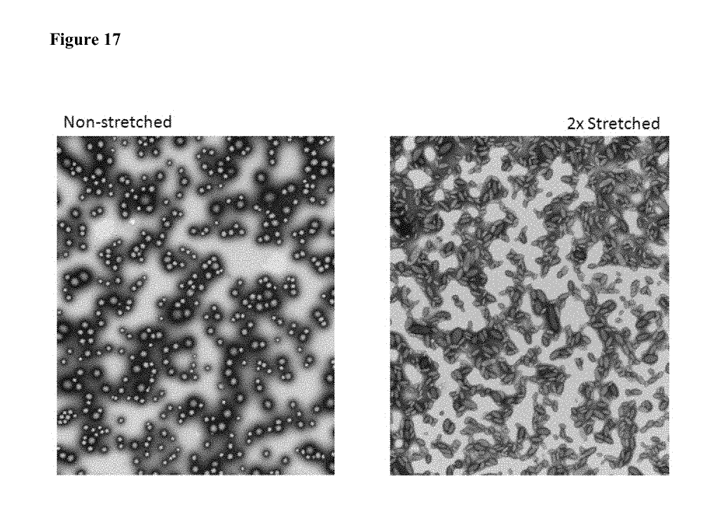

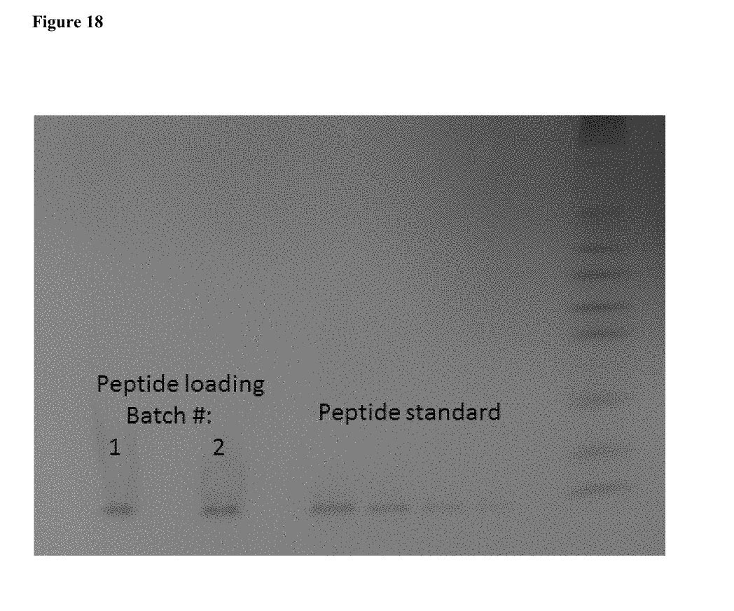

For transmission electron microscopy (TEM), nanoparticles were first resuspended in water at 1 mg/mL. 10 uL of sample was dropped onto carbon coated copper grids and left to dry in chemical hood for 2 hours. Unstained TEM imaging was then performed using the Philips CM120 system. The results are shown in FIG. 17, which shows peptide loaded NPs nonstretched (left) or 2× stretched (right).

Microparticle Loading and Release Quantification:

To measure loading, a known mass of microparticles was dissolved in DMSO. For peptide loaded microparticles, and corresponding blank microparticles, quantification was performed by running gel electrophoresis (Bio-Rad Mini-PROTEAN system) and silver stain analysis. A 12-well 10-20% Mini-PROTEAN tris-tricine gel was used, along with 10× tris/tricine/SDS running buffer diluted to 1× in Milli-Q water. Each gel contained a standard series of a known amount of peptide. The peptide standard series included 0, 62.5, 125, 250, and 500 ng of peptide per well. The remaining wells included a protein standard, and the microparticle samples, both peptide loaded and blanks as controls. The DMSO samples were mixed 1:1 by volume with sample buffer. Sample buffer was made of 24% glycerol in 1× PBS. Gel electrophoresis was run until the 2.5 kDa band of the protein standard traveled approximately two-thirds of the way down the gel. The silver stain protocol was followed for gel staining. For the development step, the stop solution was added once the lowest peptide standard (in this case 62.5 ng) began to appear. Gel images were captured with a digital camera and analyzed with ImageJ using gel band intensity quantification functionality. The results are shown in FIG. 18.

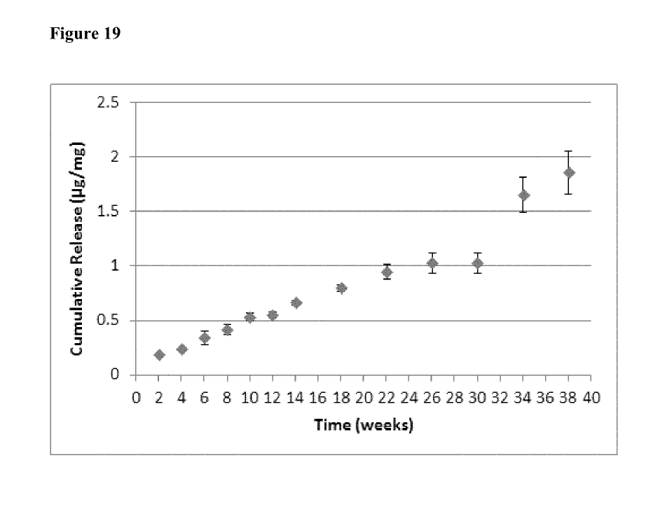

To measure release, a known mass of microparticles was suspended in 1×PBS, placed on a shaker in a 37° C. oven. At various time points, samples were centrifuged for 5 min at ˜2.5 krcf. Supernatant was collected and stored at −80° C., and fresh PBS was added to samples. Quantification of peptide released into the supernatant was performed by running gel electrophoresis, silver staining, and gel band analysis. The results are shown in FIG. 19.

1. A method for treating or preventing microvascular leakage, comprising administering an effective amount of a peptide having the amino acid sequence of any one of SEQ ID NO:1 to 6, or a derivative thereof, to a patient in need of treatment.

2. The method of claim 1, wherein the derivative is a peptide of any one of SEQ ID NOS: 7 to 31.

3. The method of claim 1, wherein the patient has or is at risk of Flu.

4. The method of claim 3, wherein the peptide is first administered within three days of first Flu symptoms.

5. The method of claim 3, wherein the peptide is first administered after first Flu symptoms.

6. The method of claim 3, wherein the peptide is first administered before first Flu symptoms.

7. The method of any one of claims 1 to 6, wherein the peptide reduces edema in the lung.

8. The method of claim 7, wherein the peptide is administered from 1 to 5 times daily.

9. The method of claim 7, wherein the peptide is administered locally to the lung.

10. The method of any one of claims 1 to 9, wherein the patient is undergoing treatment with at least one anti-viral agent and/or an anti-inflammatory agents.

11. The method of claim 1, wherein has a neuropathology associated with dysregulated angiogenesis or microvascular leakage, which is optionally MS or PD.

12. The method of claim 1, wherein the patient has Alzheimer's Disease or is identified as at risk of Alzheimer's disease, and the peptide maintains the integrity of the blood-brain barrier to thereby slow or prevent the onset or progression of Alzheimer's disease.

13. The method of claim 12, wherein the patient is undergoing treatment with at least one additional agent for treatment of Alzheimer's disease.

14. The method of any one of claims 11 to 13, wherein the peptide is administered from 1 to 5 times daily.

15. The method of claim 1, wherein the patient has or is at risk of a hemorrhagic fever.

16. The method of claim 15, wherein the patient has Ebola virus.

17. The method of claim 15, wherein the patient is undergoing treatment with at least one anti-viral agent for treatment of the hemorrhagic fever.

18. The method of any one of claims 15 to 17, wherein the peptide is administered from 1 to 5 times daily.

19. The method of claim 1, wherein the patient has cerebral malaria.

20. The method of claim 19, wherein the peptide reduces cerebral edema and/or ischemia associated with cerebral malaria.

21. The method of claim 19, wherein the patient is undergoing antimalarial therapy.

22. The method of claim 19, wherein the peptide maintains the blood brain barrier and vascular integrity in patients with cerebral malaria.

23. The method of any one of claims 19 to 22, wherein the peptide is administered from 1 to 5 times daily.

24. A method for treating cancer, comprising administering an effective amount of a peptide having the amino acid sequence of SEQ ID NO:1 to 6, or a derivative thereof, to a cancer patient undergoing or preparing to undergo therapy with an immune checkpoint inhibitor.

25. The method of claim 24, wherein the derivative is a peptide of any one of SEQ ID NOS: 7 to 31.

26. The method of claim 24, wherein the immune checkpoint inhibitor is an anti-PD-1 antibody, an anti-PD-L1 antibody, or an anti-CTLA-4 antibody.

27. The method of any one of claim 24 or 26, wherein the cancer is selected from non-small cell lung cancer, melanoma, prostate cancer, metastatic renal cell cancer.

28. The method of any one of claims 24 to 27, wherein the cancer is positive for PD-1, PD-L1 or CTLA-4.

29. The method of any one of claims 24 to 28, wherein the checkpoint inhibitor therapy is an agent that inhibits an interaction between PD-1 and PD-L1 or CTLA-4 and B7.

30. A nanoparticle comprising PLGA-PEG copolymers and a conjugated peptide targeting integrins.

31. The nanoparticle of claim 30, wherein the peptide comprises the amino acid sequence of any one of SEQ ID NOS:1 to 6, or a derivative thereof.

32. The method of claim 31, wherein the derivative is a peptide of any one of SEQ ID NOS: 7 to 31.

33. The nanoparticle of any one of claims 30 to 32, wherein the nanoparticles are formed from PLGA-PEG-peptide conjugates.

34. The nanoparticle of claim 33, wherein the nanoparticle is effective for inhibition of angiogenesis and/or lymphangiogenesis.

35. The nanoparticle of claim 33, wherein at least 50% of the polymers have conjugated peptide.

36. The nanoparticle of any one of claims 30 to 35, further comprising an encapsulated active agent.

37. The nanoparticle of claim 36, wherein the nanoparticle provides a sustained release of the active agent.

38. The nanoparticle of claim 36 or 37, wherein the active agent is a chemotherapeutic agent.

39. The nanoparticle of claim 36 or 37, wherein the active agent is a peptide agent, or targeted anti-cancer therapy.

40. The nanoparticle of any one of claims 30 to 39, having an average diameter within about 50 nm to about 500 nm, or from about 50 nm to about 100 nm.

41. The nanoparticle of any one of claims 30 to 40, wherein the nanoparticles contain an additional drug or targeting agent conjugated to the surface.

42. The nanoparticle of claim 40, wherein the nanoparticle has a zeta potential within the range of −10 to −40 mV.

43. The nanoparticle of any one of claims 30 to 42, wherein the nanoparticle is spherical.

44. The nanoparticle of any one of claims 30 to 42, wherein the particle is non-spherical.

45. A microparticle encapsulating a peptide of any one of SEQ ID NOS: 1 to 6, or derivative thereof, wherein the nanoparticle or microparticle provide a long acting depot.

46. The microparticle of claim 45, wherein the derivative is a peptide of any one of SEQ ID NOS: 7 to 31.

47. The microparticle of claim 45 or 46, wherein the particle polymers consist essentially of PLGA-PEG polymers.

48. The microparticle of any one of claim 45 or 47, wherein the particle is administered no more than once weekly or no more than once monthly.

49. The microparticle of any one of claims 45 to 48, wherein the microparticle has an average diameter in the range of about 1 μm to about 100 μm.

50. The microparticles of any one of claims 45 to 49, wherein the particles are spherical.

51. The microparticles of any one of claims 45 to 50, wherein the particles are ellipsoidal.

52. A method for treating age-related macular degeneration, diabetic macular edema, retinal vein occlusion, or diabetic retinopathy, comprising administering the nanoparticle or microparticle of any one of claims 30 to 51 to a patient in need.

53. The method of claim 52, wherein the nanoparticles or microparticles are administered by intraocular injection.

54. The method of claim 52 or 53, wherein the nanoparticles or microparticles are injected from about once daily to about monthly, to about once every six months.

55. A method for identification of integrins, comprising: contacting the nanoparticle of claim 30 with one or more cells, and visualizing or detecting binding of the nanoparticle to cells.

56. The method of claim 55, wherein the cells are in solution or in culture.

57. The method of claim 55, wherein the nanoparticle is administered to a patient, integrin over-expressing vasculature is imaged.

58. A method of treating a solid tumor, comprising administering an effective amount of the nanoparticle of any one of claims 30 to 44 to a patient in need thereof.

59. The method of claim 58, wherein solid tumor is glioblastoma or breast cancer.

60. The method of claim 59, wherein the breast cancer is triple negative breast cancer.

61. A method for treating a disease characterized by angiogenesis or vascular leakage, comprising, administering an effective amount of the nanoparticle of any one of claims 30 to 44.
